(19) United States
(12) Patent Application Publication
(10) Pub. No.: US 2005/0203708 A1
(43) Pub. Date: Sep. 15, 2005

(54) Method and system for microarray gradient detection and characterization

(76) Inventor: Srinka Ghosh, San Francisco, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/798,538

(22) Filed: Mar. 11, 2004

(51) Int. Cl. G06F 19/00; G01N 33/48; G01N 33/50

(52) U.S. Cl. 702/19

(57) ABSTRACT

A method and system for detecting and optionally characterizing background intensity gradients is disclosed. Various embodiments of the present invention employ metrics computed for a number of features within an image of a microarray that provide an indication of background intensity gradients within the image of the microarray. In one embodiment of the present invention, a metric is computed for a feature by determining the size of a region surrounding the feature for which the difference between the mean and median pixel intensities is large.

Embodiments of the present invention relate to computational analysis of microarray data and, in particular, a method and system for computationally analyzing an image of a microarray to compute metrics that allow for detection of an intensity gradient within the image of the microarray.

### BACKGROUND OF THE INVENTION

The present invention is related to microarrays. In order to facilitate discussion of the present invention, a general background for microarrays is provided, below. In the following discussion, the terms “microarray,” “molecular array,” and “array” are used interchangeably. The terms “microarray” and “molecular array” are well known and well understood in the scientific community. As discussed below, a microarray is a precisely manufactured tool which may be used in research, diagnostic testing, or various other analytical techniques.

Array technologies have gained prominence in biological research and in diagnostics. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′. end to the 3′ end, the single letter abbreviations A, T, C, and G for the nucleotide subunits that together compose the DNA polymer. For example, the oligomer 100 shown in FIG. 1 can be chemically represented as “ATCG.”

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction, or, in other words, the two strands are anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand. FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. Although deoxy-guanylate subunits of one strand are generally paired with deoxy-cytidilate subunits from the other strand, and deoxy-thymidilate subunits in one strand are generally paired with deoxy-adenylate subunits from the other strand, non-WC base pairings may occur within double-stranded DNA.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contain nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 6, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 7, with features to which labeled target molecules are hybridized similar to 706 optically or digitally differentiated from those features to which no labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

When a microarray is scanned, data may be collected as a two-dimensional digital image of the microarray, each pixel of which represents the intensity of phosphorescent, fluorescent, chemiluminescent, or radioactive emission from an area of the microarray corresponding to the pixel. A microarray data set may comprise a two-dimensional image or a list of numerical or alphanumerical pixel intensities, or any of many other computer-readable data sets. An initial series of steps employed in processing digital microarray images includes constructing a regular coordinate system for the digital image of the microarray by which the features within the digital image of the microarray can be indexed and located. For example, when the features are laid out in a periodic, rectilinear pattern, a rectilinear coordinate system is commonly constructed so that the positions of the centers of features lie as closely as possible to intersections between horizontal and vertical gridlines of the rectilinear coordinate system, alternatively, exactly half-way between a pair of adjacent horizontal and a pair of adjacent vertical grid lines. Then, regions of interest (“ROIs”) are computed, based on the initially estimated positions of the features in the coordinate grid, and centroids for the ROIs are computed in order to refine the positions of the features. Once the position of a feature is refined, feature pixels can be differentiated from background pixels within the ROI, and the signal corresponding to the feature can then be computed by integrating the intensity over the feature pixels.

In general, the intensity associated with a pixel in the image of a microarray is the sum of: (1) a signal-intensity component produced, at a location of the surface of the microarray corresponding to the pixel, by bound target molecules; and (2) a background-intensity component produced by a wide variety of background-intensity-producing sources, including noise produced by electronic and optical components of a microarray scanner, general non-specific reflection of light from the surface of the microarray during scanning, or, in the case of radio-labeled target molecules, natural sources of background radiation, and various defects and contaminants on, and damage associated with, the surface of the microarray. A wide variety of different computational techniques are employed to determine the background-intensity components of pixel intensities and to subtract the background-intensity components from measured pixel intensities in order to recover the signal-intensity components. Unfortunately, it may be difficult to precisely determine the background intensity components of pixels in an image of a microarray, particularly when the microarray contains contaminants and/or defects that produce background intensity gradients within the image of the microarray. For this reason, designers, manufacturers, and users of microarrays and microarray scanners continue to seek improved and computationally efficient methods for detecting defects and damage reflected in background intensity gradients in images of microarrays.

### SUMMARY OF THE INVENTION

Various embodiments of the present invention may employ metrics computed for a number of features within an image of a microarray that provide an indication of background intensity gradients within the image of the microarray. In one embodiment of the present invention, a convergence metric is computed for a feature by determining the size of a region surrounding the feature for which the difference between the mean and median pixel intensifies is large. In one or more embodiments, features with computed metrics of large magnitudes are generally found in, or adjacent to, regions within the image of the microarray with steep background intensity gradients. The presence of features with computed metrics of large magnitudes may be used in one or more embodiments as an indication of the presence of background intensity gradients within the image of the microarray, and the patterns of distribution of such features within an array of features may be used to provide an indication of the location and directions of background intensity gradients within the image of the microarray.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIGS. 4-7 illustrate the principle of array-based hybridization assays.

FIGS. 8, 9, and 10 illustrate problems associated with intensity gradients within images of microarrays.

FIG. 11 shows a small portion of the image of a microarray that includes a central feature and four nearest neighbor features.

FIG. 12 shows the subregion of a scanned image of a microarray of FIG. 11 with numerical intensities associated with the central feature pixels and the background pixels within three annular backgrounds regions.

FIG. 13 illustrates the subregion of an image of a microarray illustrated in FIGS. 11 and 12 with a background-intensity gradient running through the subregion.

FIG. 14 illustrates calculation of the convergence metric χ for the central features of the subregions illustrated in FIGS. 12 and 13.

FIG. 15 illustrates a characteristic plot of average minus median background-pixel intensities for annular backgrounds of increasing radius about a feature within a region of an image of a microarray having a significant intensity gradient.

FIGS. 16 and 17 illustrate tracking of a region of contamination within a subregion of an image of a microarray by computed convergence metrics for features within the subregion of an image of the microarray.

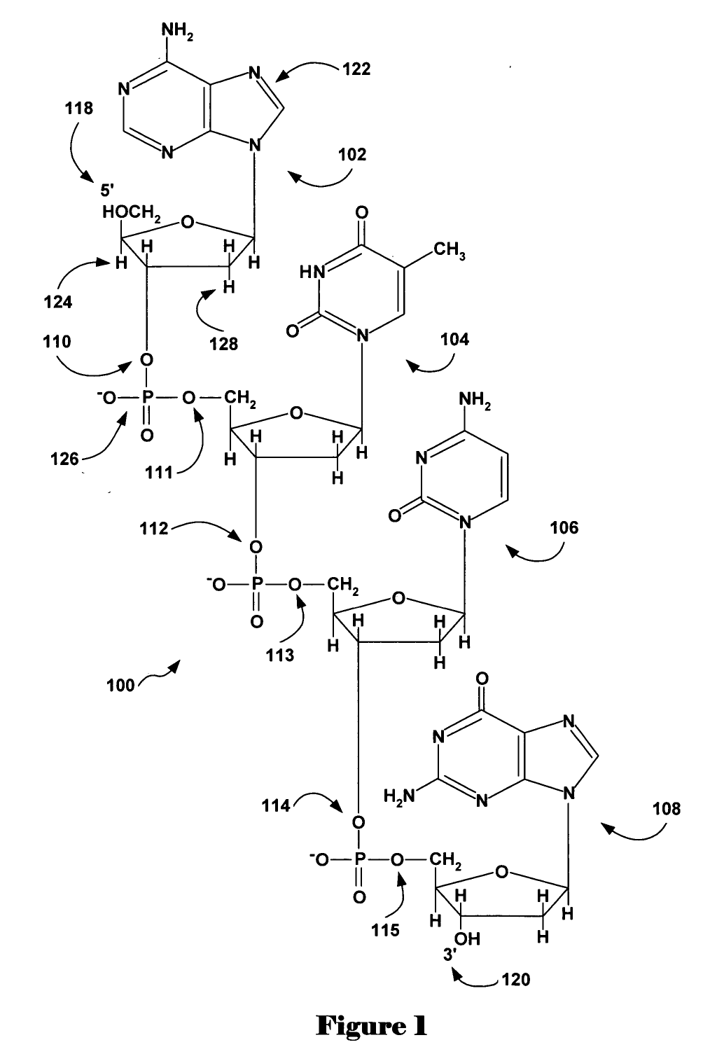

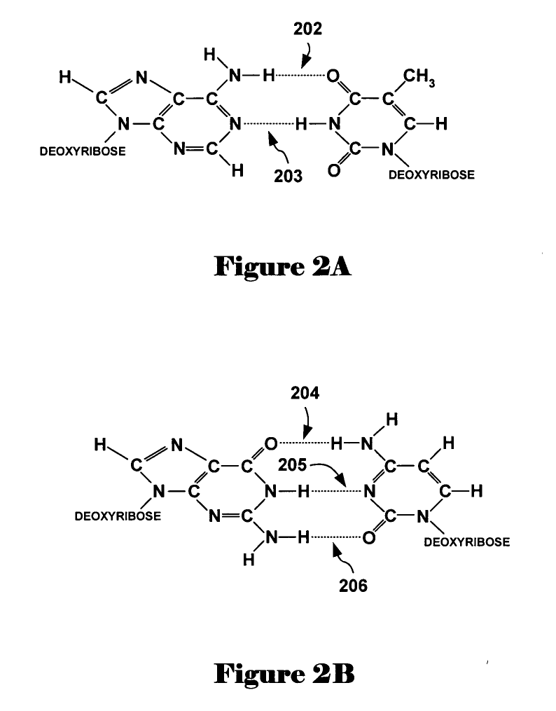

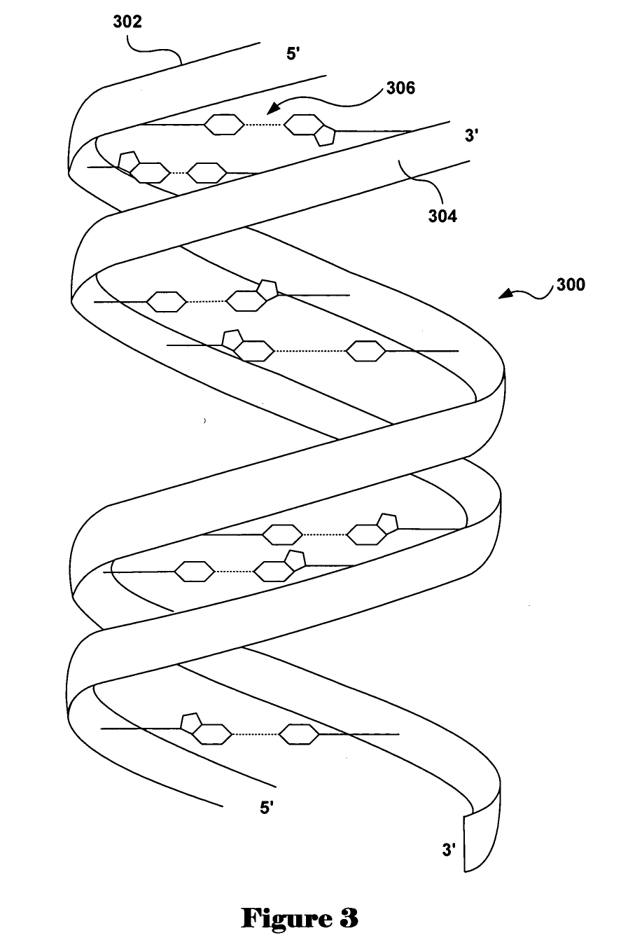

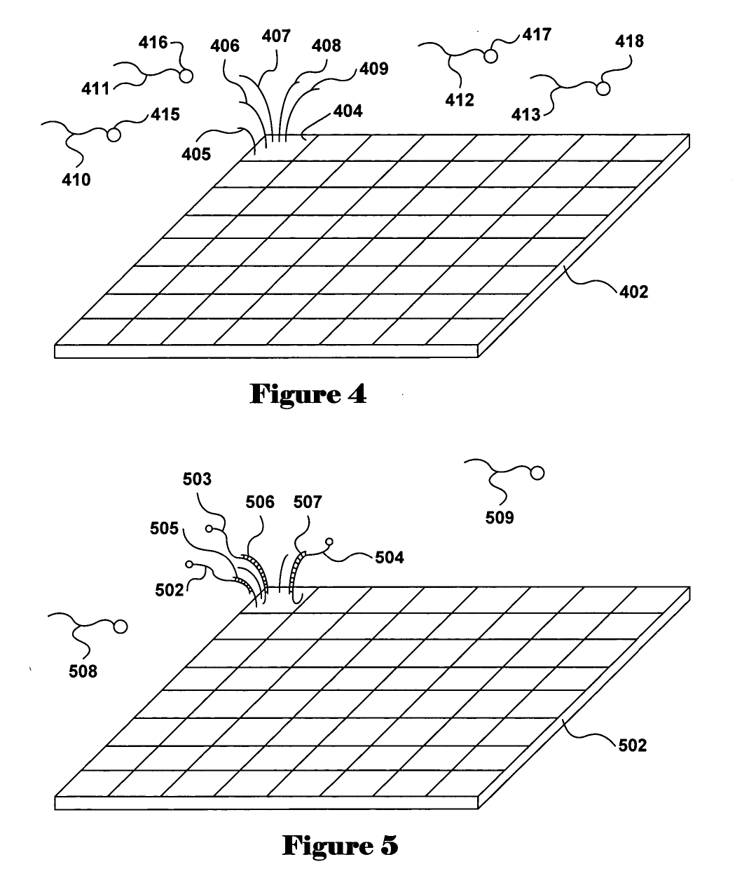

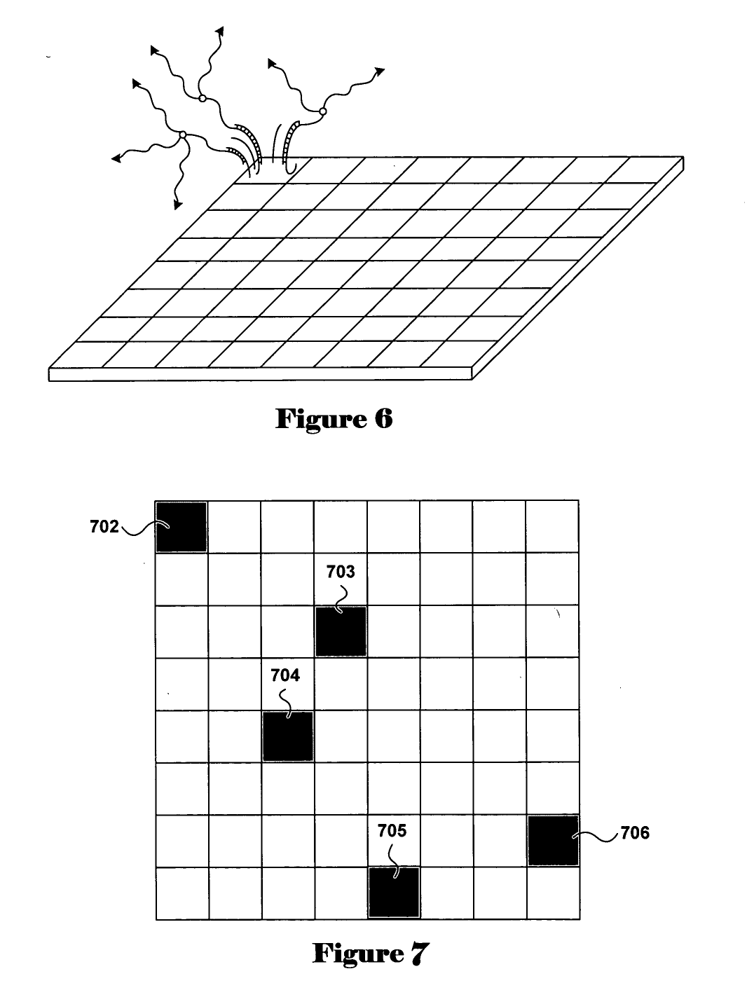

### DETAILED DESCRIPTION OF THE INVENTION

The present invention relates to methods and systems for detecting the presence of intensity gradients and characterizing intensity gradients within images of microarrays. In a first subsection, below, additional information about microarrays is provided. Next, in a second subsection, embodiments of the present invention are described with reference to a number of figures that illustrate simple examples. Finally, in a third subsection, a C++-like pseudocode implementation of a method for computing convergence-metrics is provided as a detailed illustration of one embodiment of the present invention.

### Additional Information About Microarrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Inter-feature areas are typically, but not necessarily, present. Inter-feature areas generally do not carry probe molecules. Such inter-feature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

A molecular array is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in published U.S. patent applications 20030160183A1, 20020160369A1, 20040023224A1, and 20040021055A, as well as U.S. Pat. No. 6,406,849. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array, followed by application of a method of the present invention, may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces an image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular-array data can produce detailed chemical and biological analyses, disease diagnoses, and other information that can be stored in a computer-readable medium, transferred to an intercommunicating entity via electronic signals, printed in a human-readable format, or otherwise made available for further use.

### Embodiments of the Present Invention

In general, a pixel intensity within an image of a microarray is expected to be the sum of a signal-intensity component, generated by a bound target molecule on the surface of a microarray, and a background-intensity component, generated by various background-intensity sources, as discussed above. The background intensity is expected to be relatively uniformly distributed within the image of a microarray, and have a variance and average magnitude characteristic for the type of microarray, experimental procedure, and microarray scanner used to scan the microarray. In processing microarray data, the background-intensity component is generally estimated, by any of various computational techniques, and subtracted from the measured intensity to produce an estimated signal intensity. However, in reality, the background intensity components associated with pixels in the image of a microarray may be quite non-uniformly distributed. Non-uniform distributions of background-pixel intensities across the image of a microarray may result from background intensity gradients within the image of a microarray. For example, a disk-shaped region of contamination on the surface of a microarray may lead to a corresponding disk-shaped region, in the image of a microarray, with much greater or lower background intensities. An intensity gradient within the image of the microarray is found within an annular band of pixels containing the circumference of the disk-shaped region of contamination, with directions of steepest increase or decrease of background intensities within the gradient corresponding to the intersections of radial lines emanating from the center of the disk-shaped region of contamination and the annular gradient. Similarly, the image of a microarray partly dipped into a contaminating solution may show a background intensity gradient near the line dividing the portion of the microarray immersed in the contaminating solution and the portion of the array not immersed in the contaminating solution, and therefore free of contamination. Manufacturing defects, thumbprints, scratches, and other such defects and damage to the surface of a microarray may produce multiple, complicated gradients within the image of a microarray.

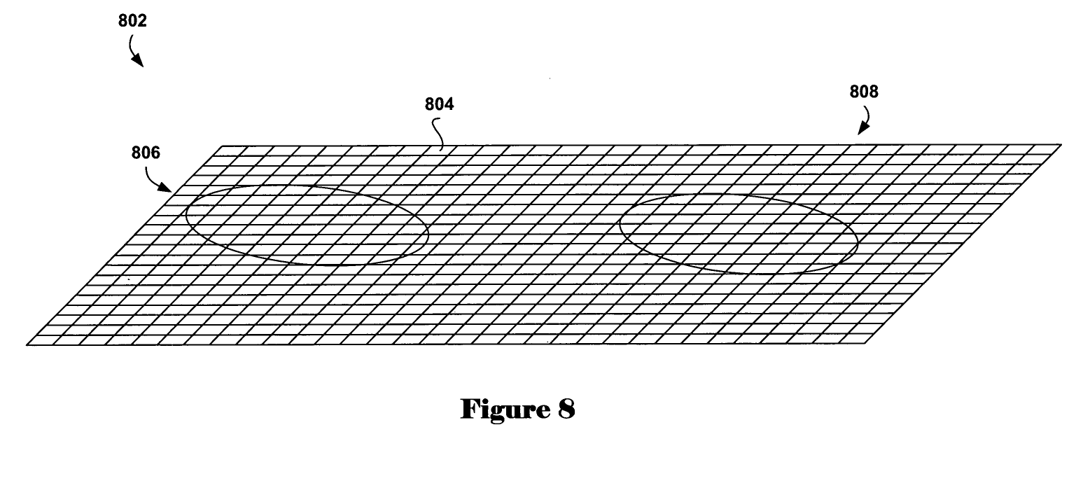

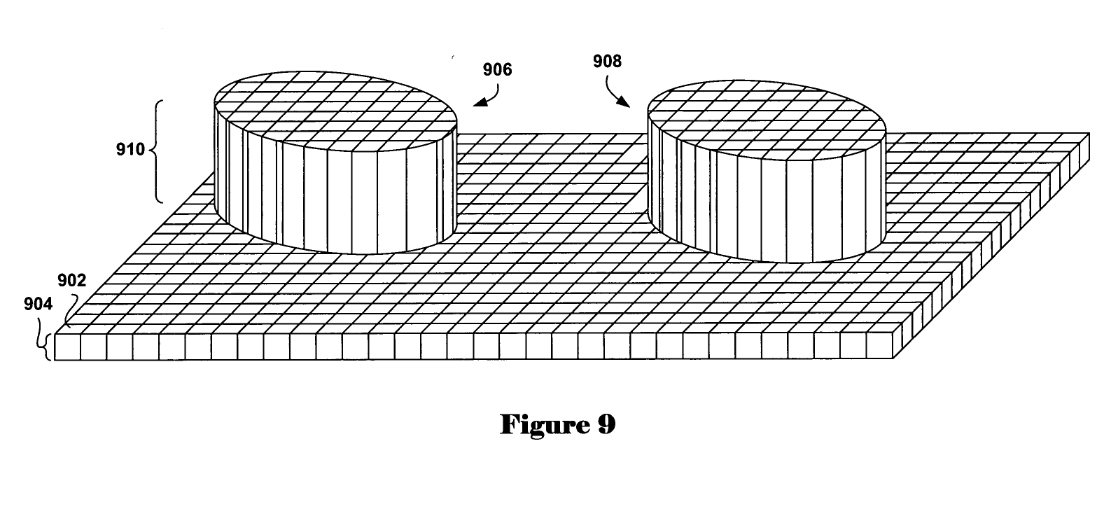

FIGS. 8, 9, and 10 illustrate problems associated with intensity gradients within images of microarrays. FIG. 8 shows a small, rectangular region 802 of a pixel-based image of a microarray. Each pixel is represented as a small square region, such as pixel 804, bounded by grid lines. The subregion of an image of a microarray shown in FIG. 8 includes two disk-shaped regions 806 and 808 that contain contiguous pixels associated with intensities, each intensity comprising a signal intensity component and a background intensity component. Pixels outside of the disk-shaped regions 806 and 808 are associated with intensities composed solely of background intensity components. FIG. 9 illustrates pixel intensities within the subregion of the image of the microarray shown in FIG. 8. In FIG. 9, the intensities associated with pixels within a rectilinear grid in the x and y directions are represented as the heights, in the z direction, of the rectangular volumes corresponding to the pixels. For example, the intensity measured for pixel 902 is represented as the height 904 of the rectangular volume with base corresponding to the area of the pixel in the image of a microarray. In FIG. 9, all of the pixels of the illustrated subregion have identical background-intensity components, and, therefore, the heights of the non-features pixels in the subregion are identical. The rectangular volumes corresponding to the non-feature pixels form a planar surface in the FIG. 9 at a height in the z direction equal to the background-intensity components associated with the pixels. The feature pixels in FIG. 9 appear as two columns 906 and 908 rising above the plane of the background pixels by a height equal to the signal-intensity components of the feature pixels. The height 910 of the columns 906 and 908 above the plane of the background pixels therefore represents the signal-intensity components of the feature pixels. In a normal image of a microarray, the background-intensity components and signal-intensity components of pixel intensities vary about a mean, and therefore the surfaces of the background-pixel volume elements and feature-pixel volume elements in FIG. 9 would be non-planar, with the heights of the rectangles representing the pixels varying about mean values. However, for clarity of illustration, the signal-intensity components of feature pixels and the background-intensity components of both feature and non-feature pixels have identical values in the example shown in FIG. 9. When the background intensity components are uniformly distributed within the image of a microarray, the signal-intensity components of pixels may be easily obtained by subtracting the uniform, background-intensity component, determined from the non-feature pixels, from the intensities of the feature pixels.

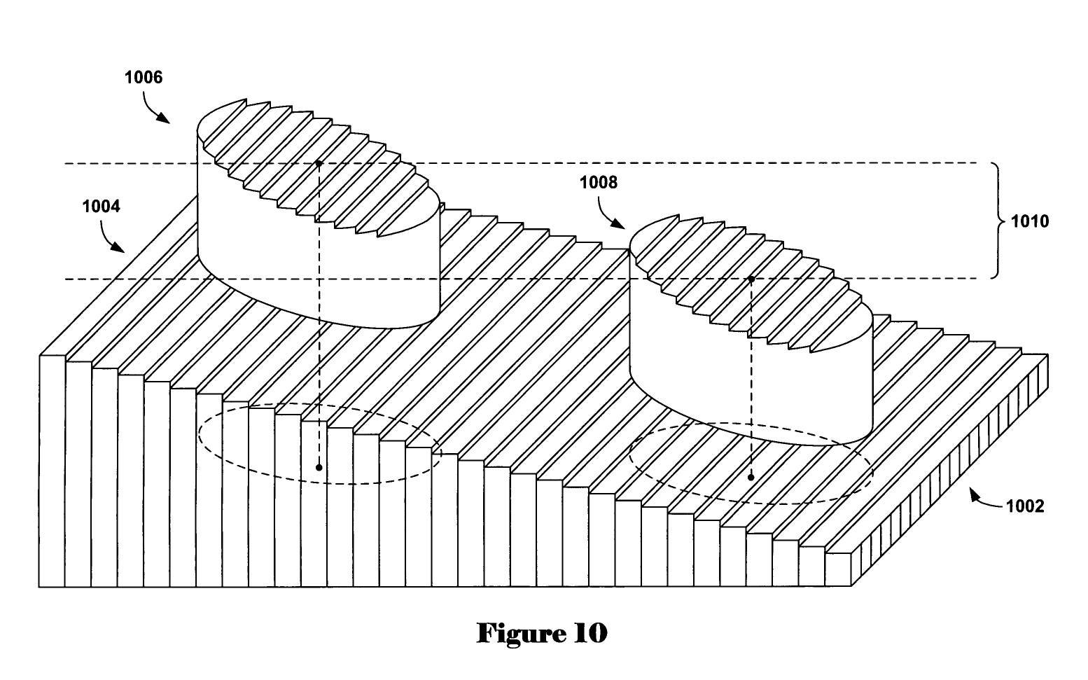

FIG. 10 illustrates the subregion of the image of a microarray shown in FIG. 8 with a rather steep background-intensity component gradient along a horizontal direction of the subregion. As shown in FIG. 10, the intensities associated with non-feature pixels increase from relatively small values 1002 at the right-hand edge of the subregion to relatively large values 1004 at the left-hand edge of the subregion. As in FIG. 9, the signal-intensity components of the intensities associated with feature pixels are identical for the two features in the subregion illustrated in FIG. 10. However, because of the steep background-intensity component gradient within the subregion, the intensities for the pixels associated with the left-hand feature 1006 are greater than the intensities associated with pixels of the right-hand feature 1008. Moreover, the intensities of pixels within a single feature also vary in the direction of the background-intensity gradient, so that the upper surface of the columns 1006 and 1008 representing the feature-pixel intensities are steeply sloped. Therefore, even though the signal-intensity components of the pixels associated with both features are identical, the intensities of the pixels associated with the left-hand feature 1006 are significantly greater, by an intensity difference 1010, than the intensities associated with the right-hand feature pixels.

In order to carry out background subtraction to recover feature signals from a microarray data set, the presence of gradients, such as the gradient illustrated in the subregion of FIG. 10, need to be detected and characterized. For a simple linear gradient, as shown in FIG. 10, many currently available feature-extraction methods can be used to obtain accurate integrated signal intensities by detecting, quantifying, and correcting for the linear background intensity gradient across the image of the microarray. However, simple gradients represent only a small subset of the different types of intensity gradients observed in images of microarrays. Complexly shaped manufacturing defects, patterns of contamination, thumbprints and fingerprints, and scratches and abrasions may produce intricately shaped, non-continuous regions of intensity gradients, with varying directions of steepest increase, within the image of a microarray. In many cases, currently available feature-extraction programs have difficulty detecting and correcting for such complex gradients.

Embodiments of the present invention are directed to computationally inexpensive techniques for detecting and characterizing intensity gradients within the image of a microarray. In these embodiments, a metric is computed for a number of features within the image of the microarray based on non-feature, background regions surrounding the number of features. Those features associated with computed metrics of large magnitudes are often found in regions of the image of a microarray with significant intensity gradients. The distributions of features within the image of the microarray with computed metrics of high magnitudes are indicative of the locations of intensity gradients within the image of the microarray.

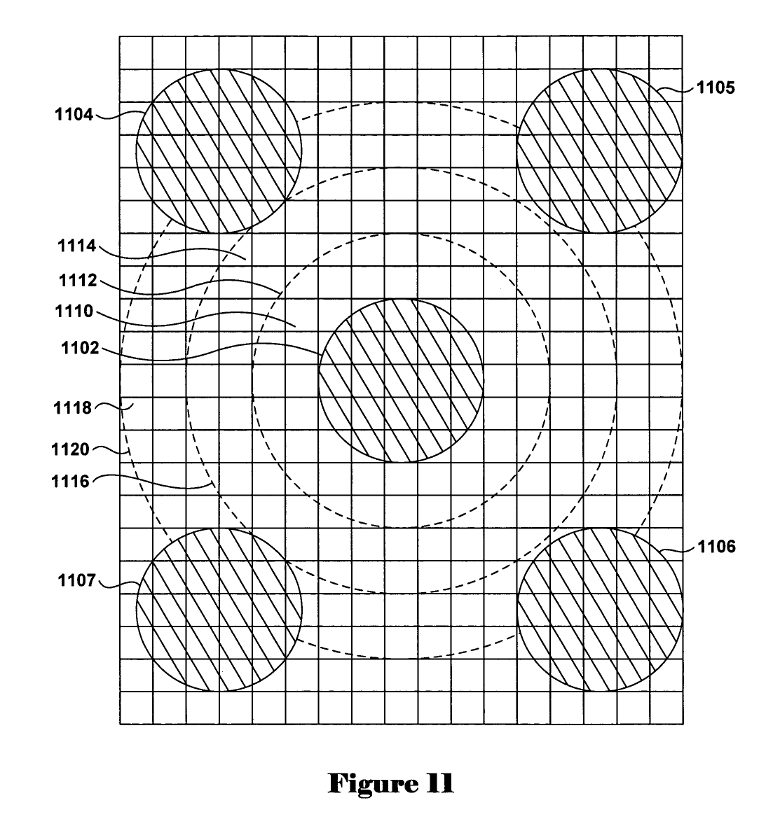

FIG. 11 shows a small portion of the image of a microarray that includes a central feature and four nearest neighbor features. In FIG. 11, the central feature 1102 and the four nearest neighbor features 1104-1107 are represented as disk-shaped regions with crosshatching. In FIG. 11, the central feature 1102 is surrounded by annular regions containing non-feature pixels of increasing radii. For example, the central feature 1102 is surrounded by a first annular region 1110 with an outer radius 1112 of 4.5 pixels. The central feature 1102 is surrounded by a second, annular background region 1114 having an outer radius 1116 of 6.5 pixels. The second annular background region 1114 includes the first annular background region 1110. Similarly, the central feature 1102 has a third annular background region 1118 of outer radius 1120. The third annular background region 1118 includes the second annular background region 1114 which in turn includes the first annular background region 1110. Feature-pixels both from the central feature and from neighboring features are excluded from the annular background regions of increasing radii about the particular feature.

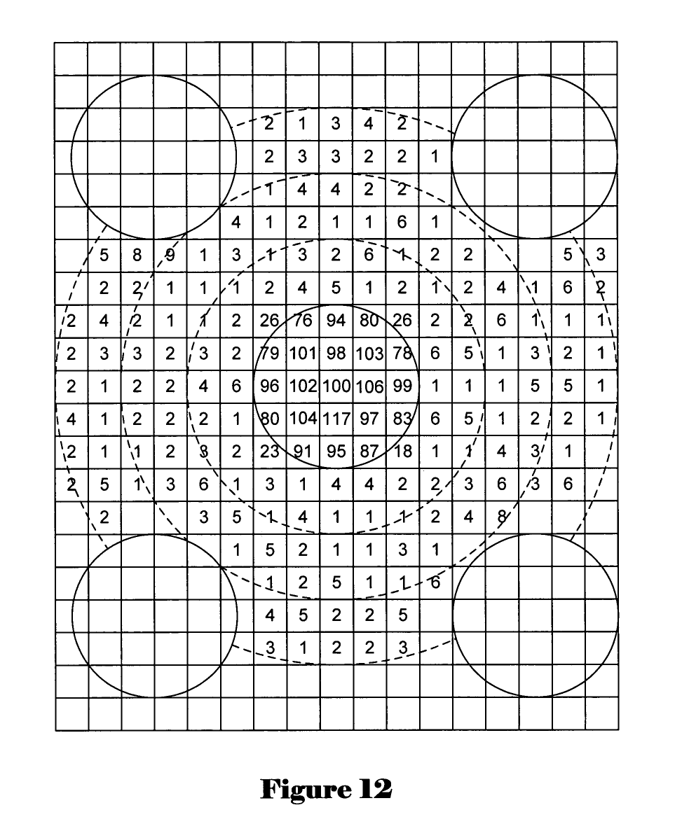

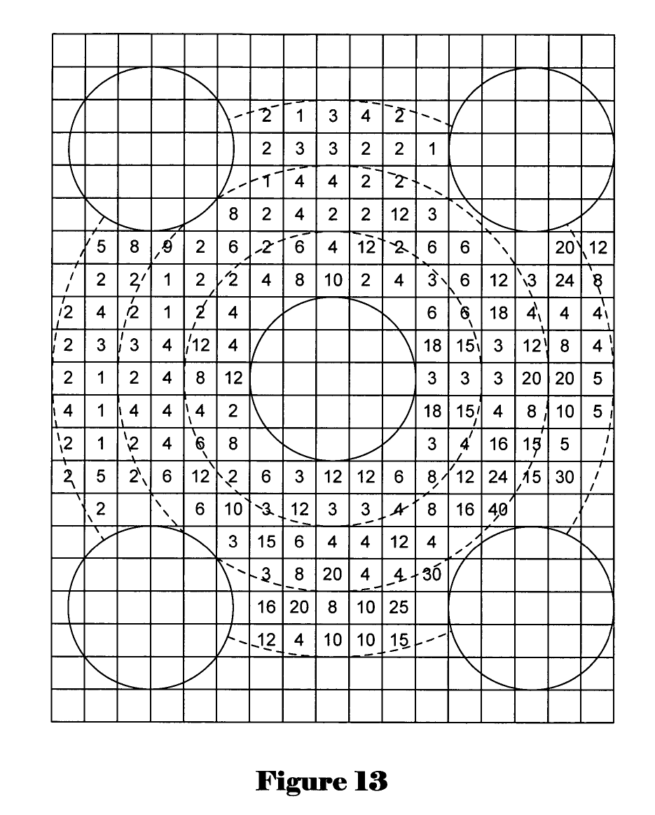

FIG. 12 shows the subregion of an image of a microarray of FIG. 11 with numerical intensities associated with the central feature pixels and with the background pixels of the three annular backgrounds regions. In the subregion shown in FIG. 12, the central-feature pixel intensities have relatively high intensity values distributed about an average central-feature-pixel intensity, and the background pixels have relatively low intensity values distributed about a relatively low, average background-pixel intensity. FIG. 13 illustrates the subregion of an image of a microarray illustrated in FIGS. 11 and 12 with a background-intensity gradient running through the subregion. As can be seen in FIG. 13, background-pixel intensities towards the lower, right-hand corner of the subregion are greater than background-pixel intensities near the upper, left-hand corner of the subregion.

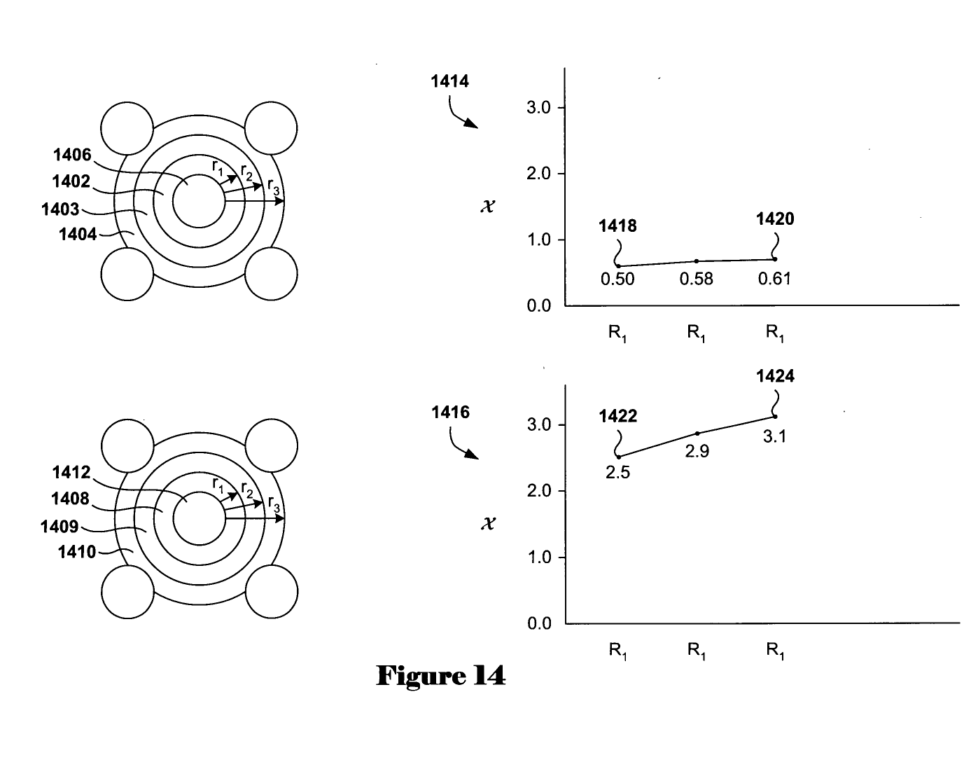

One useful metric that can be calculated for selected features within the image of a microarray is the convergence metric χ. FIG. 14 illustrates calculation of the convergence metric χ for the central features of the subregions illustrated in FIGS. 12 and 13. The convergence metric χ is defined as the outer radius of the annular background region surrounding a disk-shaped feature, the semimajor or semiminor axis length of the outer ellipse of an elliptical background region surrounding an elliptical disk-shaped feature, or the half width of a rectangular background region surrounding a rectangular shaped feature, having the greatest difference between a computed average background-pixel intensity and the median background-pixel intensity within the annular or rectangular background median. Alternative convergence metrics are possible, including converge metrics related to the above-described convergence metrics by constant multipliers, as well as the lengths of other types of mathematical features correlated with background region areas. The convergence metric χ is computed by computing the difference between the mean and median background-pixel intensities in background regions of increasing radius or half width about a particular feature, and then selecting as the convergence metric χthe radius of the background region for which the computed difference between the background-pixel average intensity and background-pixel median intensity is greatest. However, when the average/median-background-pixel-intensity difference computed for background regions of increasing radius or half widths do not show significant increase or decrease, then the convergence metric χ is defined to be the radius or half width of the feature. For example, in FIG. 14, the differences between the average background-pixel intensity and the median background-pixel intensity within the three annular background regions of increasing radius 1402-1404 about the central feature 1406 in the subregion illustrated in FIG. 12 are plotted in graph 1414, and the differences between the average background-pixel intensity and median background-pixel intensity for the annular background regions 1408-1410 surrounding central feature 1414 in the subregion illustrated in FIG. 13 are plotted in graph 1416. In graphs 1414 and 1416, the vertical, y axis corresponds to the difference between. the average and median background-pixel intensities, and the horizontal, or x axis corresponds to the outer radius of the annular background regions. As can be seen in graph 1414, the difference between the average background-pixel intensity and median background-pixel intensity for the three annular background regions of increasing radius about the central feature 1406 of the subregion shown in FIG. 12 range from a value of 0.50 (1418) to a value of 0.61 (1420). Similarly, the difference between the average background-pixel intensities and the median background-pixel intensities for the three annular background regions of increasing radius 1408-1410 surrounding the central feature 1412 of the subregion shown in FIG. 13 are plotted in graph 1416 and range from a value of 2.5 (1422) to a value of 3.1 (1424). For the subregion shown in FIG. 12, without a background-intensity gradient, the differences between the average and median background-pixel intensities computed for the annular background regions of increasing radius are relatively constant, and of small magnitude. In this case, since the differences neither appreciably decrease nor increase, the convergence metric χ is defined to be the radius of the central feature 1406, or 2.5 pixels. By contrast, for the subregion shown in FIG. 13 with a significant background-intensity gradient, the differences between the average and mean background-pixel intensities have relatively large magnitude and steadily increase over the range of background-annulus radii. In this case, the convergence metric χ is defined to be the radius of the third, largest annular background region r₃. Convergence metrics computed for features within regions of an image of a microarray without significant intensity gradients are generally equal to, or close to, the feature radii, while convergence metrics computed for features within the image of a microarray in regions with significant intensity gradients are generally significantly larger than the feature radii.

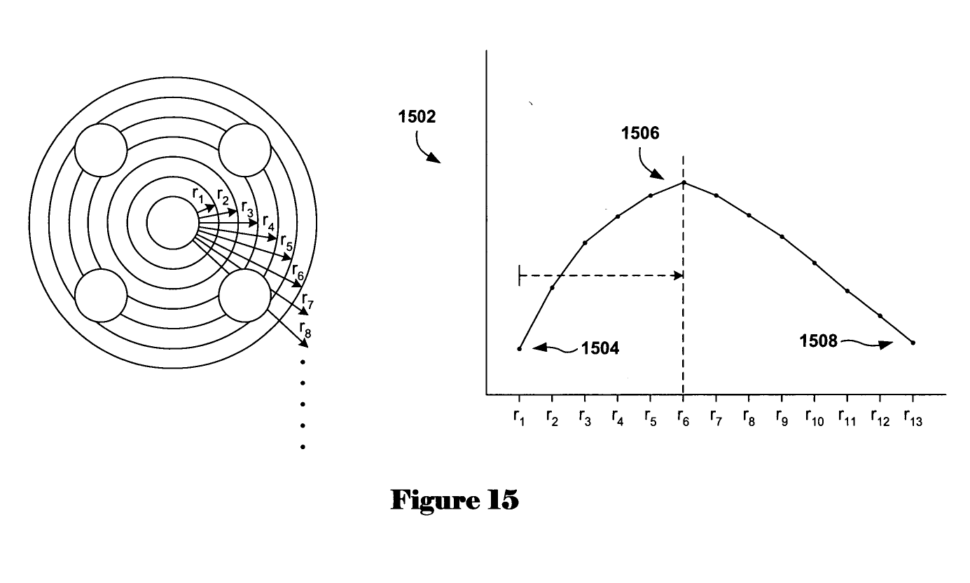

FIG. 15 illustrates a characteristic plot of average minus median background-pixel intensities for annular backgrounds of increasing radius about a feature within a region of the image of a microarray having a significant intensity gradient. FIG. 15 uses the same illustration conventions used in FIG. 14. As shown in FIG. 15, the plot of the difference between the average and median background-pixel intensities for the annular background regions of increasing radii r₁-r₁₃ 1502 produces a curve that rises from a relatively low, initial value 1504 to a peak value 1506 and then falls back to a relatively low value 1508. In this case, the convergence metric χ would be computed as the outer radius r₆ of the annular background region exhibiting the greatest difference between the average and median background-pixel intensities. In general, the convergence metric χ represents a measure of the relative size of the intensity gradient within which a feature is embedded.

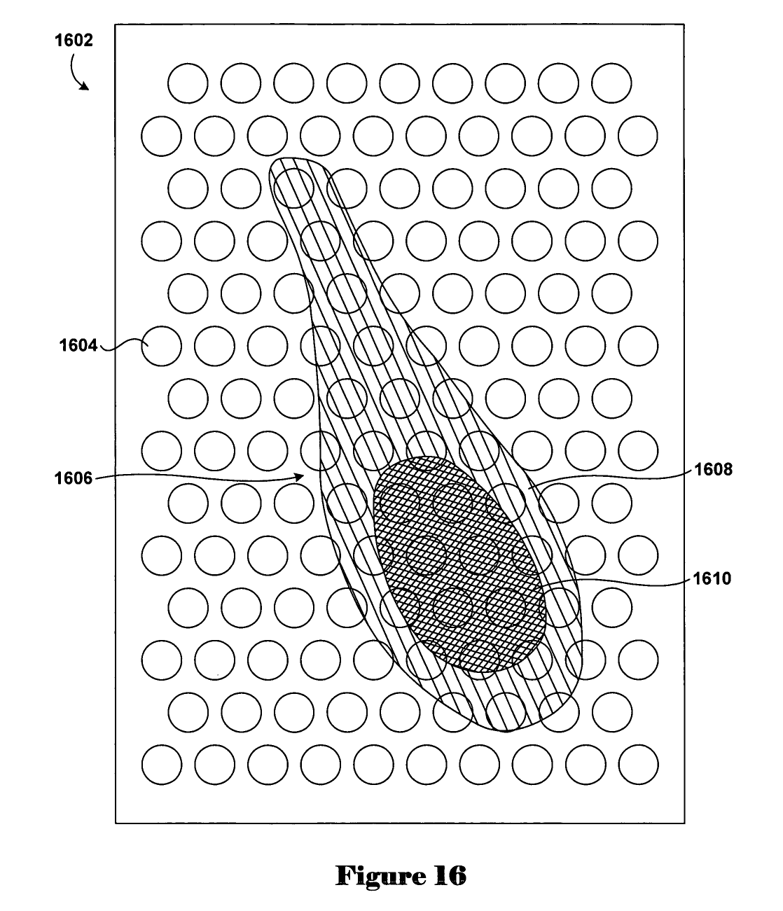

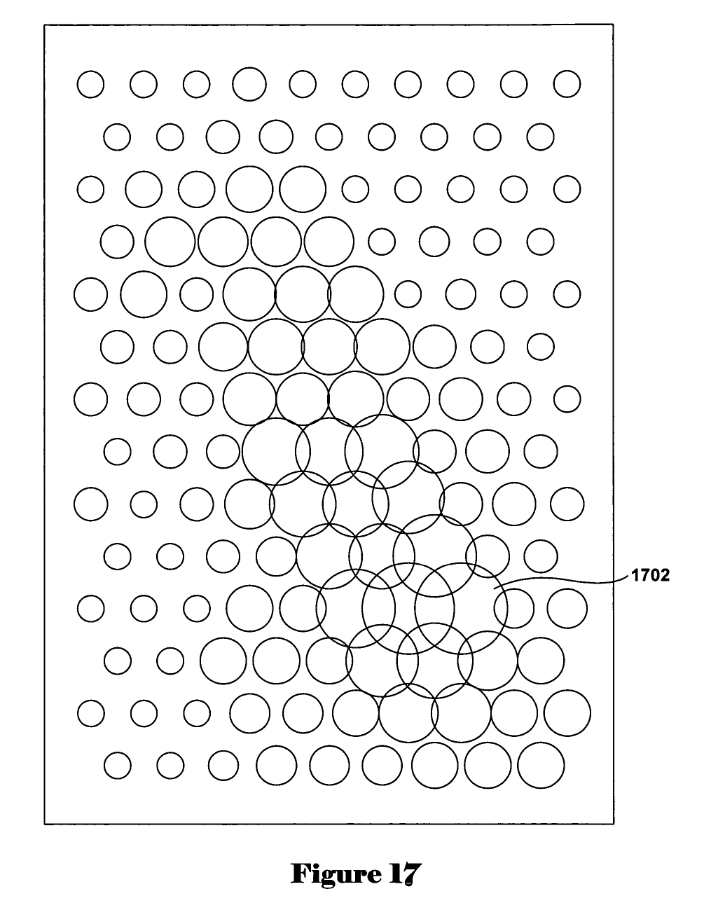

FIGS. 16 and 17 illustrate tracking of a region of contamination within a subregion of an image of a microarray by computed convergence metrics for features within the subregion of the image of the microarray. As shown in FIG. 16, the subregion of an image of the microarray 1602 includes a number of disk-shaped features, such as feature 1604. Within the subregion, a large, amorphously shaped region of contamination 1606 is present. The region of contamination 1606 includes an outer region 1608 of moderate contamination and an inner region 1610 with high contamination. The degree of contamination is directly related to the much higher background intensity for the region of contamination 1606 in the image of the microarray. FIG. 17 shows the same subregion of the image of the microarray shown in FIG. 16 with the convergence metrics for each feature shown as circles with radii equal to the computed convergence metrics. As can be seen in FIG. 17, features with relatively large convergence metrics, such as feature 1702, correspond to features within the contaminated region (1606 in FIG. 16), especially features at the boundaries between regions of significantly different average background intensities. Thus, the computed convergence metrics for features in the image of a microarray both indicate the presence of intensity gradients within the image of the microarray as well as provide indications of the locations, shapes, and background-intensity magnitudes of the regions in the image of the microarray having significant intensity gradients.

### C++-Like Pseudocode Implementation of an Embodiment of the Present Invention

In this subsection, a relatively straightforward, C++-like pseudocode implementation of one embodiment of the present invention is provided. This pseudocode implementation is provided for illustrative purposes only. It is not, for example, intended to limit the scope of the present invention to the particular implementation details discussed below. As with any software routine, there are an almost limitless number of possible implementations for any given embodiment.

First, a number of constant declarations are provided:
1 const int HEIGHT=100;
2 const int WIDTH=100;
3 const int MaxIntensity=255;
4 const int Maxincrements=10;
5 const int Increment=3;
6 const int maxH=10;
7 const int maxW=10;
8 const double Threshold=3;
9 const int Feature_x_spacing=15;
10 const int Feature_y_spacing=15;
11 const int Feature_radius=5;
12 const int Feature00_x=9;
13 const int Feature00_y=9;
14 const int aMaskDim=((Feature_radius+MaxIncrements*Increment)*2)+1;
15 const int ai=aMaskDim/2;
16 const int aj=ai;
   The constants HEIGHT and WIDTH, declared above on lines 1-2, define the maximally sized microarray image, in pixels, handled by the implementation. In actual images of microarrays, the dimensions of the image, in pixels, may be thousands, tens of thousands, or hundreds of thousands of pixels. The constant “MaxIntensity,” declared above on line 3, defines the range of possible pixel intensities. In the illustrative pseudocode, only 256 intensity values are used, although an actual image of a microarray may use 16-bit, 32-bit, or larger integer sizes for storing intensity values, and may therefore support a much larger number of possible intensities. In such cases, an implementation different from that provided in the pseudocode may be used. The constant “MaxIncrements,” declared above on line 4, defines the maximum number of annular background regions of increasing radii used to compute the convergence metric χ for a feature within the image of a microarray. In alternative implementations, a much larger number may be employed. The constant “Increment,” declared above on line 5, defines the width, in pixels, of an annular background region. In other words, the difference in outer diameters of adjacent annular background regions is three pixels. The constants “maxH” and “maxW,” declared above on lines 6-7, define the maximum size, in features, of the feature array embedded within the image of the microarray. The constant “Threshold,” declared above on line 8, is a threshold difference between the average and median background-pixel intensities calculated for a series of annular backgrounds of increasing radius that determines whether the convergence metric χ is assigned the feature radius, when the difference is below the threshold value, or the radius of the annular background region with the greatest difference between average and median background-pixel intensities. The constants “Feature_x_spacing” and “Feature_y_spacing,” declared above on lines 9-10, define the spacings between features in an image and the horizontal and vertical directions, respectively. The constant “Feature_radius,” declared above on line 11, defines the radius, in pixels, for the features in the microarray. Note that, in many implementations, a design file describing the locations and sizes of features within the image of a microarray is normally employed, rather than depending on a simple, constant-defined grid array, as in the current pseudocode implementation. The simple approach is employed in this implementation to facilitate clarity of illustration. The constants “Feature00_x” and “Feature00_y” declared above on lines 12-13, define the position of the topmost, right-hand feature with feature index (0,0) in the image of the microarray. The constants “aMaskDim,” “ai,” and “aj,” declared above on lines 14-16, define the dimension of an annulus mask that is used to compute the average and median intensity within annular backgrounds surrounding a given feature. In the described embodiment, the annulus mask is square, and a single dimension defines its size. The constants “ai” and “aj” are the pixel coordinates of the central pixel in the annulus mask.

Next, a single class declaration is provided as scaffolding for a handful of methods that implement one embodiment of the present invention:

| | |
|---|---|
| 1 | class scannedImage |
| 2 | { |
| 3 | private: |
| 4 | unsigned char image[HEIGHT][WIDTH]; |
| 5 | unsigned char annulusMask[aMaskDim][aMaskDim]; |
| 6 | int pixels[MaxIntensity][MaxIncrements]; |
| 7 | double meansMinusMedians[MaxIncrements]; |
| 8 | unsigned char radii[maxH][maxW]; |
| 9 | int maxX; |
| 10 | int maxY; |
| 11 | |
| 12 | void computeFeatureIndexes( ); |
| 13 | int distance(int px, int py, int pxc, int pyc); |
| 14 | bool translateFeatureToPixel(int x, int y, int & px, |
| | int & py); |
| 15 | void computeAnnulusMask( ); |
| 16 | void clearData( ); |
| 17 | int computeAnnuli(int x, int y, int & low, int & high); |
| 18 | unsigned char computeRadiusOfConvergence(int x, int y); |
| 19 | |
| 20 | public: |
| 21 | void computeRadiiOfConvergence( ); |
| 22 | scannedImage( ); |
| 23 | }; |

The class “scannedlmage” includes the following private data members: (1) “image,” declared above on line 4, a two-dimensional byte array representing an image of a microarray, each byte in the two-dimensional array representing the intensity associated with a pixel in the image; (2) “annulusMask,” declared above on line 5, a two-dimensional byte array that contains numeric values representing the membership of corresponding pixels within background annuli surrounding a central feature, within the central feature, or within features near the central feature, the numeric value “0” indicating a feature pixel, and numeric values greater than the value “0”indicating the number of an annulus in a sequence of annuli extending outward from the central feature; (3) “pixels,” declared above on line 6, a two-dimensional array indexed by potential pixel intensity values and the sequence number for annular backgrounds surrounding a central feature, which stores the number of pixels of each intensity value within each annular background region surrounding the feature; (4) “meansMinusMedians,” declared above on line 7, an array containing the computed difference between the average and median background-pixel intensities within annular background regions of increasing radii; (5) “radii,” a two-dimensional array declared above, on line 8, that contains the computed convergence metric □ for each feature in the microarray; and (6) “maxX” and “maxY,” declared above on lines 9-10, which contain the maximum horizontal and vertical feature indices for the image of the microarray.

The class “scannedImage” includes the following private data members: (1) “computeFeatureIndexes,” declared above on line 12, which computes the convergence metrics for all the features within the image of the microarray; (2) “distance,” declared above on line 13, which computes the distance between two pixels in a rectilinear grid, or array, of pixels, the two pixels specified by x and y coordinates supplied as arguments; (3) “translateFeatureToPixel,” declared above on line 14, which translates feature coordinates (x,y) to pixel coordinates (px,py); (4) “computeAnnulusMask,” declared above on line 15, which prepares the private data member “annulusMask” for use in subsequently computing convergence metrics for the features in the image of the microarray; (5) “clearData,” declared above on line 16, that clears the contents of the private data members “pixels” and “meansMinusMedians” prior to computing the convergence metric χ for a particular feature; (6) “computeAnnuli,” declared above on line 17, which counts the number of pixels of each possible intensity within all of the background annuli about a feature at feature coordinates (x,y), supplied as arguments; and (7) “computeRadiusOfConvergence,” declared above on line 18, which computes the convergence metric χ for a feature at feature coordinates (x,y), supplied as arguments. The class “scananedImage” includes the public function member “computerRadiiOfConvergence,” declared above on line 21, that computes the convergence metrics for all features within the image of a microarray and stores the computed convergence metrics in the two-dimensional array “radii,” and a constructor, which obtains the image of a microarray and stores the pixel intensity values in the private data member “image.”

An implementation of the function member “computeFeatureIndexes” is next provided:

| | |
|---|---|
| 1 | void scannedImage::computeFeatureIndexes( ) |
| 2 | { |
| 3 | int featureWidth = WIDTH − (2 * Feature00_x); |
| 4 | int featureHeight = HEIGHT − (2 * Feature00_y); |
| 5 | |
| 6 | maxX = (featureWidth / Feature_x_spacing) + 1; |
| 7 | maxY = (featureHeight / Feature_y_spacing) + 1; |
| 8 | } |

The function member “computeFeatureIndexes” determines values for the private data members “maxX” and “maxY,” described above. These private data members indicate the dimensions of a feature array contained within the scanned image of the microarray.

An implementation of the function member “distance” is next provided:

| | |
|---|---|
| 1 | int scannedImage::distance(int px, int py, int pxc, int pyc) |
| 2 | { |
| 3 | double diffX, diffY; |
| 4 | double dist; |
| 5 | |
| 6 | diffX = px − pxc; |
| 7 | diffY = py − pyc; |
| 8 | diffX *= diffX; |
| 9 | diffY *= diffY; |
| 10 | dist = sqrt(diffX + diffY); |
| 11 | return dist; |
| 12 | } |

The function member “distance” determines the distance, in pixels, of a pixel with coordinates (px, py) from a pixel with coordinates (pxc, pyc). The distance is determined by a simple Euclidean distance function.

An implementation of the function member “translateFeatureToPixel” is next provided:

1 bool scannedImage::translateFeatureToPixel(int x, int y, int & px,
int & py)
2{
3 if (maxW < x ∥ maxH < y) return false;
4 px = (x * Feature_x_spacing) + Feature00_x;
5 py = (y * Feature_y_spacing) + Feature00_y;
6 return true;
7 }

The function member “translateFeatureToPixel” translates the position of a feature, specified in feature coordinates (x,y), into pixel coordinates (px, py). The feature spacings and position of the corner feature (0,0) within the image of the microarray, specified by the constants “Feature_x_spacing,” “Feature_y_spacing,” “Feature00_x,” and “Feature00_y,” are used to translate the feature coordinates (x,y), into pixel coordinates (px, py).

An implementation of the function member “computeAnnulusMask” is next provided:

| | |
|---|---|
| 1 | void scannedImage::computeAnnulusMask( ) |
| 2 | { |
| 3 | |
| 4 | int i, j; |
| 5 | int m, n, p, q; |
| 6 | int d; |
| 7 | int rem; |
| 8 | bool negx, negy; |
| 9 | |
| 10 | for (i = 0; i < aMaskDim; i++) |
| 11 | for (j = 0; j < aMaskDim; j++) |
| 12 | { |
| 13 | d = distance(i, j, ai, aj); |
| 14 | if (d <= 5) annulusMask[i][j] = 0; |
| 15 | else annulusMask[i][j] = ((d − Feature_radius − 1) / |

Increment) + 1;

| | |
|---|---|
| 16 | m = i − ai; |
| 17 | if (m < 0) |
| 18 | { |
| 19 | negx = true; |
| 20 | m = −m; |
| 21 | } |
| 22 | else negx = false; |
| 23 | p = m / Feature_x_spacing; |
| 24 | rem = m % Feature_x_spacing; |
| 25 | if (rem >= (Feature_x_spacing) >> 1) p++; |
| 26 | if (negx) p = ai − (p * Feature_x_spacing); |
| 27 | else p = ai + (p * Feature_x_spacing); |
| 28 | n = j − aj; |
| 29 | if (n < 0) |
| 30 | { |
| 31 | negy = true; |
| 32 | n = −n; |
| 33 | } |
| 34 | else negy = false; |
| 35 | q = n / Feature_y_spacing; |
| 36 | rem = n % Feature_y_spacing; |
| 37 | if (rem >= (Feature_y_spacing) >> 1) q++; |
| 38 | if (negy) q = aj − (q * Feature_y_spacing); |
| 39 | else q = aj + (q * Feature_y_spacing); |
| 40 | d = distance(i, j, p, q); |
| 41 | if (d <= 5) annulusMask[i][j] = 0; |
| 42 | } |
| 43 | } |

The function member “computeAnnulusMask” generates an annulus mask, stored in the private data member “annulusMask,” that is used to determine whether pixels n the neighborhood of a feature belong to the feature, to a neighboring feature, or to one of a number of concentric annular background regions surrounding the feature. The center of the mask is superimposed on the image of the microarray at the center of a feature in order to select background pixels in the neighborhood of the feature. Feature pixels are represented by the value “0,” while background pixels have a numeric value equal to or greater than “1,” indicating the largest background annulus to which the pixel belongs. In the nested for-loops of lines 10-43, each mask position is indexed by the loop variables i and j. A value is determined for each position. On line 13, the distance between the position (i,j) and the center of the annulus mask is determined. On lines 14-15, the value for position (ij) is set to “0,” if the position is within a distance equal to the feature radius of the center of the annulus mask, and otherwise set to a value equal to the number of background annulus widths that the position is distant from the feature, plus 1. On lines 23-39, the pixel coordinates (p,q) of the nearest feature center to the position (i,j) is determined, and then, on line 40, the distance of the position (i,j) from the center of the nearest feature is determined. If that distance is less than or equal to the feature radius, the value corresponding to the position in the annulus mask is set to 0on line 41.

An implementation of the function member “clearData” is next provided:

| | |
|---|---|
| 1 | void scannedImage::clearData( ) |
| 2 | { |
| 3 | int i,j; |
| 4 | |
| 5 | for (i = 0; i < MaxIntensity; i++) |
| 6 | { |
| 7 | for (j = 0; j < MaxIncrements; j++) |
| 8 | pixels[i][j] = 0; |
| 9 | } |
| 10 | for (j = 0; j < MaxIncrements; j++) |
| 11 | meansMinusMedians[j] = 0; |
| 12 | } |

The function member “clearData” simply clears the arrays “pixels” and “meansMinusMedians” to prepare for computation of a convergence metric □ for a next feature.

Next, an implementation of the function member “computeAnnuli” is provided:

| | |
|---|---|
| 1 | int scannedImage::computeAnnuli(int pxc, int pyc, int & low, |
| | int & high) |
| 2 | { |
| 3 | int i, j; |
| 4 | int left, right, bottom, top; |
| 5 | unsigned char intensity; |
| 6 | unsigned char annulus; |
| 7 | int smallestD = 10000; |
| 8 | int d; |
| 9 | int maxI; |
| 10 | |
| 11 | low = 255; |
| 12 | high = 0; |
| 13 | left = pxc − ai; |
| 14 | if (left < 0) |
| 15 | { |
| 16 | if (pxc < smallestD) smallestD = pxc; |
| 17 | left = 0; |
| 18 | } |
| 19 | right = pxc + ai; |
| 20 | if (right >= WIDTH) |
| 21 | { |
| 22 | d = WIDTH − pxc; |
| 23 | if (d < smallestD) smallestD = d; |
| 24 | right = WIDTH − 1; |
| 25 | } |
| 26 | top = pyc − aj; |
| 27 | if (top < 0) |
| 28 | { |
| 29 | if (pyc < smallestD) smallestD = pyc; |
| 30 | top = 0; |
| 31 | } |
| 32 | bottom = pyc + aj; |
| 33 | if (bottom >= HEIGHT) |
| 34 | { |
| 35 | d = HEIGHT − pyc; |
| 36 | if (d < smallestD) smallestD = d; |
| 37 | bottom = HEIGHT − 1; |
| 38 | } |
| 39 | |
| 40 | if (smallestD < 10000) |
| 41 | { |
| 42 | left = pxc − smallestD; |
| 43 | right = pxc + smallestD; |
| 44 | top = pyc − smallestD; |
| 45 | bottom = pyc + smallestD; |
| 46 | maxI = (smallestD − Feature_radius) / Increment; |
| 47 | } |
| 48 | else maxI = MaxIncrements; |
| 49 | for (i = left; i <= right; i++) |
| 50 | { |
| 51 | for (j = top; j <= bottom; j++) |
| 52 | { |
| 53 | annulus = annulusMask[i − (pxc − ai)][j − (pyc − aj)]; |
| 54 | if (annulus > 0 && annulus < maxI) |
| 55 | { |
| 56 | intensity = image[i][j]; |
| 57 | if (intensity < low) low = intensity; |
| 58 | if (intensity > high) high = intensity; |
| 59 | pixels[intensity][annulus − 1]++; |
| 60 | } |
| 61 | } |
| 62 | } |
| 63 | return maxI; |
| 64 | } |

The function member “computeAnnuli” computes and stores the counts of the number of pixels with each possible pixel intensity into the two-dimensional array “pixels.” The array “pixels” is indexed both by possible intensity values and by the annulus in which the intensities are counted. Note that, in the case of function member “computeAnnuli,” the pixel intensity counts are for strict annuli, rather than annuli that include all annuli of smaller radius. The function member “computeAnnuli” keeps track of the lowest and highest intensities observed in the variable arguments “low” and high.” On lines 13-48, the function member “computeAnnuli” determines the range of positions in the annulus mask that overlap image pixels when the center of the annulus map is coincident with the feature at pixel coordinates (pxc, pyc) passed to the function member “computeAnnuli” as arguments. Then, in the nested for-loops of lines 49-62, the function member “computeAnnuli” considers each position of the annulus mask overlapping the image in the neighborhood of the feature at pixel coordinates (pxc, pyc), and tabulates the umber of times each possible intensity value is observed in each strict background annulus. Note that only intensities for pixels within annuli in the overlap region are tabulated.

Next, an implementation of the function member “computeRadiusOfConvergence” is provided:

| | |
|---|---|
| 1 | unsigned char scannedImage::computeRadiusOfConvergence(int x, |
| | int y) |
| 2 | { |
| 3 | int pxc, pyc; |
| 4 | int i, j, k, m, n; |
| 5 | int cnt; |
| 6 | double num; |
| 7 | double sum, isum; |
| 8 | double avg; |
| 9 | double max = −1000; |
| 10 | double min = 1000; |
| 11 | int low, high; |
| 12 | double res; |
| 13 | int maxI; |
| 14 | |
| 15 | if (!translateFeatureToPixel(x, y, pxc, pyc)) return |
| | Feature_radius; |
| 16 | clearData( ); |
| 17 | maxI = computeAnnuli(pxc, pyc, low, high); |
| 18 | |
| 19 | for (i = 0; i < maxI; i++) |
| 20 | { |
| 21 | num = 0; |
| 22 | sum = 0; |
| 23 | if (i > 0) |
| 24 | { |
| 25 | n = i − 1; |
| 26 | for (m = low; m <= high; m++) |
| 27 | { |
| 28 | pixels[m][i] += pixels[m][n]; |
| 29 | cnt = pixels[m][i]; |
| 30 | num += cnt; |
| 31 | sum += m * cnt; |
| 32 | } |
| 33 | } |
| 34 | else |
| 35 | { |
| 36 | for (m = low; m <= high; m++) |
| 37 | { |
| 38 | cnt = pixels[m][i]; |
| 39 | num += cnt; |
| 40 | sum += m * cnt; |
| 41 | } |
| 42 | } |
| 43 | if (num < 30) |
| 44 | { |
| 45 | i−−; |
| 46 | break; |
| 47 | } |
| 48 | isum = 0; |
| 49 | for (m = low;; m++) |
| 50 | { |
| 51 | isum += pixels[m][i]; |
| 52 | if (isum >= num / 2) break; |
| 53 | } |
| 54 | avg = sum / num; |
| 55 | res = avg − m; |
| 56 | if (res < 0) res = −res; |
| 57 | meansMinusMedians[i] = res; |
| 58 | } |
| 59 | if (i < 2) return Feature_radius; |
| 60 | for (j = 0; j < i; j++) |
| 61 | { |
| 62 | res = meansMinusMedians[j]; |
| 63 | if (res > max) |
| 64 | { |
| 65 | k = j; |
| 66 | max = res; |
| 67 | } |
| 68 | if (res < min) min = res; |
| 69 | } |
| 70 | if (max − min < Threshold) return Feature_radius; |
| 71 | else return Feature_radius + (k * Increment); |
| 72 | } |

The function member “computerRadiusOfConvergence” computes a convergence metric χ for the feature at feature coordinates (x,y), passed to the function member “computerRadiusOfConvergence” as arguments. First, on lines 15-17, the feature coordinates (x,y) are translated to pixel coordinates (pxc, pyc) and the number of times each possible intensity is observed for each strict background annulus is tabulated via a call to function member “computeAnnuli.” Then, in for-loop of lines 19-58, the mean and median pixel intensities for each background annulus of increasing radius, as described with reference to FIGS. 12-15, are computed. First, either in the for-loop of lines 26-32 or 36-41, the number of pixels considered in the annulus is determined, as well as a sum of all pixel intensities. The for-loop of lines 26-32 is used for all annuli greater than the initial annulus, and the intensity counts for the strict annulus, stored in the two-dimensional array “pixels” is added to the counts for the preceding annulus. On lines 48-53, the median intensity for the currently considered annulus is computed. The mean intensity is computed on line 54. The absolute value of the difference between the mean and median pixel intensity for the currently considered annulus is then determined on lines 55-56. The absolute value of the difference between the mean and median pixel intensity for the currently considered annulus is then stored into the array “meansMinusMedians” on line 57. In the for-loop of lines 60-69, the array “meansMinusMedians” is searched for the largest absolute value of the difference between the mean and median pixel intensity, and the radius of the corresponding annulus is returned as the convergence metric χ on line 71, if the difference between the largest and smallest values stored in the array “meansMinusMedians” is greater than the value “Threshold.”

An implementation of the function member “computerRadiiOfConvergence” is next provided:

| | |
|---|---|
| 1 | void scannedImage::computeRadiiOfConvergence( ) |
| 2 | { |
| 3 | int i, j; |
| 4 | |
| 5 | computeFeatureIndexes( ); |
| 6 | computeAnnulusMask( ); |
| 7 | for (i = 0; i < maxX; i++) |
| 8 | { |
| 9 | for (j = 0; j < maxY; j++) |
| 10 | radii[i][j] = computeRadiusOfConvergence(i, j); |
| 11 | } |
| 12 | } |

The above function computes the convergence metric χ for each feature in the image of the microarray via a call to function member “computeRadiusofconvergence,” and stores each computed convergence metric χ into the two-dimensional array “radii.”

Once convergence metrics are computed for all or a selected number of features within a microarray, the computed convergence metrics can be used in many different ways. For example, the computed convergence metrics may be sorted, and the number of computed convergence metrics with magnitudes greater than the threshold magnitude determined in order to decide whether or not the image of the microarray contains a sufficient proportion of features within intensity gradients to make the microarray unsuitable for feature extraction. Alternatively, the features within the image of a microarray with computed convergence metrics greater than a threshold value may be clustered together based on position in order to identify subregions within the image of the microarray with high intensity gradients, and those subregions may either be considered unsuitable for feature extraction, or may be specially treated or flagged by feature extraction programs.

Although the present invention has been described in terms of a particular embodiment, it is not intended that the invention be limited to this embodiment. Modifications within the spirit of the invention will be apparent to those skilled in the art. For example, many different possible convergence metrics may be used to identify features within regions containing intensity gradients. In the above-described implementation, the absolute value of the difference between the average and median intensities of features within background annuli are computed to find the annulus with the greatest absolute value of difference between average and median pixel intensities. However, different, similar metrics are possible. For example, the square of the computed difference may be employed. In the above-described implementation, the annulus with the greatest absolute value of the difference between average and median pixel intensities is selected to define the convergence metric, but in alternative implementations, an annulus immediately preceding or following the annulus with the greatest difference between average and median pixel intensities may instead be selected. The selection may also be based on the shape of the plotted difference versus radii graphs, described above with reference to FIGS. 14 and 15, may be based on the derivatives of that curve, or on a variety of different numerical and computational values based on pixel intensities within annular background regions of increasing radii. As another example, a statistical value, such as the variance or standard deviation of pixel intensities may be used, rather than the difference between the average and median pixel intensities. As discussed above, square background regions of increasing half widths may be employed for square features, and a variety of other different shaped background regions of increasing dimensions may be employed where appropriate. As mentioned above, there are an almost limitless number of implementations of a convergence metric computation routine. Different control structures, data structures, programming languages, and numerical techniques may be employed to compute the convergence metric. The above pseudocode implementation is implemented for clarity of illustration, rather than efficiency of computation. Ordinarily skilled programmers would be expected to be able to improve the computational efficiency of a convergence metric routine or routines by standards techniques, including performance profiling followed by application of computationally efficient techniques where needed to improve the computational efficiency of the routines.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for detecting a background intensity gradient within a microarray data set, the method comprising:
computing metrics for features within the microarray data set; and
when the metrics computed for a number of features are larger than a threshold value, determining that the microarray data set exhibits a background intensity gradiant.

2. The method of claim 1 wherein a metric is computed for each feature in the microarray data set.

3. The method of claim 1 wherein a metric is computed for a selected number of features in the microarray data set.

4. The method of claim 1 wherein the metric computed for each feature is a metric related to a difference between mean and median pixel intensities within background regions of increasing size containing the feature.

5. The method of claim 4 wherein the metric computed for each feature is proportional to a size of a background region containing the feature with a greatest difference between the mean and median pixel intensity for pixels within the background region.

6. The method of claim 4 wherein the metric computed for a feature is proportional to a size of a background region containing the feature with a greatest difference between the mean and median pixel intensity for pixels within the background region, when a difference between a largest difference between mean and median pixel intensity for pixels within a background region and a smallest difference between mean and median pixel intensity for pixels within a background region is greater than a threshold value, and otherwise the metric computed for a feature is a size of the feature.

7. The method of claim 4 wherein the metric computed for each feature is proportional to a size of a background region containing the feature with a difference between the mean and median pixel intensity for pixels within the background region near to, but not equal to, the size of a background region with a greatest difference between the mean and median pixel intensity for pixels within the background region.

8. The method of claim 4 wherein features are disk shaped, and the background regions of increasing size are annuli circumscribing the feature with increasing outer radii.

9. The method comprising forwarding, to a remote location an indication obtained by a method of claim 1 as to whether or not a microarray data set contains a background intensity gradient.

10. The method comprising receiving from a remote location an indication obtained by a method of claim 1 as to whether or not a microarray data set contains a background intensity gradient.

11. A computer program implementing the method of claim 1 stored in a computer-readable medium.

12. A method for characterizing background intensity gradients within a microarray data set, the method comprising:
computing metrics for features within the microarray data set; and
when the metrics computed for a number of features are larger than a threshold value,
grouping features with computed metrics by position; and
characterizing a background intensity gradient corresponding to each group of features by an area of the microarray surface corresponding to the group and by a position of the group on the surface of the microarray.

13. The method of claim 12 further including characterizing a background intensity gradient corresponding to a group of features by an average computed metric for the features of the group.

14. The method of claim 12 wherein a metric is computed for each feature in the microarray data set.

15. The method of claim 12 wherein a metric is computed for a selected number of features in the microarray data set.

16. The method of claim 12 wherein the metric computed for each feature is a metric related to a difference between mean and median pixel intensities within background regions of increasing size containing the feature.

17. The method of claim 16 wherein features are disk shaped, and the background regions of increasing size are annuli circumscribing the feature with increasing outer radii.

18. The method of claim 12 further comprising forwarding, to a remote location a characterization of a background intensity gradient within the microarray data set.

19. A computer program implementing the method of claim 12 stored in a computer-readable medium.

20. A microarray data set analysis system comprising:
a stored image of a microarray; and
a processing entity that
computes a metric for features within the image of the microarray; and when the metrics computed for a number of features are larger than a threshold value,
determines that a background intensity gradient is present in the image of the microarray;
groups features with computed metrics larger than a threshold value by position; and
characterizes a background intensity gradient corresponding to each group of features.

21. The microarray data set analysis system of claim 20 wherein the processing entity characterizes the background intensity gradient corresponding to each group of features by one or more of:
an area of the microarray surface corresponding to the group of features;
a position of the group of features on the surface of the microarray; and
an average computed metric for the group of features.

22. The microarray data set analysis system of claim 20 wherein a metric is computed for each feature in the image of the microarray.

23. The microarray data set analysis system of claim 20 wherein a metric is computed for a selected number of features in the image of the microarray.

24. The microarray data set analysis system of claim 20 wherein the metric computed for each feature is a metric related to a difference between mean and median pixel intensities within background regions of increasing size containing the feature.

25. The microarray data set analysis system of claim 24 wherein features are disk shaped, and the background regions of increasing size are annuli circumscribing the feature with increasing outer radii.
